(19) United States
(12) Patent Application Publication
(10) Pub. No.: US 2009/0311317 A1
(43) Pub. Date: Dec. 17, 2009

(54) MODIFIED RELEASE TOLTERODINE FORMULATIONS

(75) Inventors: Subraman Rao CHERUKURI, Vienna, VA (US); Venkat N. RAVELLA, Gandhinagar (IN)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Capricorn Pharma Inc., Frederick, MD (US)

(21) Appl. No.: 12/466,359

(22) Filed: May 14, 2009

Related U.S. Application Data
(60) Provisional application No. 61/127,523, filed on May 14, 2008.

(51) Int. Cl. A61K 9/54; A61K 31/137; A61P 25/00

(52) U.S. Cl. 424/456; 514/648

(57) ABSTRACT

Modified or extended release formulations containing tolterodine and associated methods are disclosed and described. Methods for making and administering said modified release formulations are also disclosed.

### CROSS-REFERENCE TO RELATED APPLICATIONS

This application claims the benefit of U.S. Provisional Application Ser. No. 61/127,523, filed May 14, 2008, which is incorporated by reference in its entirety herein.

### FIELD OF THE INVENTION

The present invention relates to tolterodine containing formulations with desired in-vitro and in-vivo characteristics which are simple to formulate and economical to manufacture on a commercial scale. Accordingly, the present invention involves the field of pharmaceutical sciences.

### BACKGROUND OF THE INVENTION

A substantial part (5-10%) of the adult population suffers from overactive or unstable urinary bladder, often also referred to as urinary incontinence. The symptoms of an unstable or overactive bladder comprise urge incontinence, urgency and urinary frequency. The prevalence of overactive bladder, particularly of so-called urge incontinence, increases with age. It is assumed that unstable or overactive bladder is caused by uncontrolled contractions of the bundles of smooth muscle fibers forming the muscular coat of the urinary bladder (the detrusor muscle) during the filling phase of the bladder. These contractions are mainly controlled by cholinergic muscarinic receptors, and the pharmacological treatment of unstable or overactive bladder has been based on muscarinic receptor antagonists.

Recently an improved muscarinic receptor antagonist, tolterodine tartarate, also known as (+)-N,N-diisopropyl-3-(2-hydroxy-5-methylphenyl)-3-phenylpropylamine L-hydrogen tartrate (structure of tolterodine free base shown below), has been marketed for the treatment of urge incontinence and other symptoms of unstable or overactive urinary bladder.

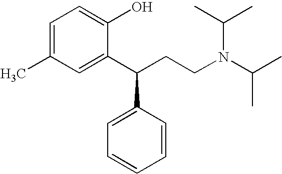

Both tolterodine and its major, active metabolite, the 5-hydroxymethyl derivative of tolterodine, significantly contribute to the therapeutic effect. Tolterodine and it's 5-hydroxymethyl derivative have considerably less side-effects than another drug used to treat overactive or unstable urinary bladder, oxybutynin, especially regarding the propensity to cause dry mouth. This is because although tolterodine is equipotent with oxybutynin in the bladder, its affinity for muscarinic receptors of the salivary gland is eight times lower than that of oxybutynin. See, e.g., Nilvebrant, L., et al., European Journal of Pharmacology 327: 195-207 (1997). The selective effect of tolterodine in humans is described in Stahl, M. M. S., et al., Neurourology and Urodynamics 14: 647-655 (1995) and Bryne, N., International Journal of Clinical Pharmacology and Therapeutics 35: 287-295 (1995). Accordingly, tolterodine is amore attractive drug for the treatment of overactive or unstable urinary bladder.

### SUMMARY OF THE INVENTION

One of the advantages presented by the claimed tolterodine dosage forms is that the claimed dosage forms provide a homogeneous composition, that is optionally coated, that contains a precise amount of tolterodine. Moreover, there is very little variability from dosage form to dosage form (e.g., from pellet to pellet) in the claimed tolterodine dosage forms in terms of the biopharmaceutical behavior (e.g., dissolution, drug deliver, and drug absorption) between dosage forms (e.g., pellet to pellet). Accordingly, the claimed dosage tolterodine dosage forms are ideal for the delivery of a low dosage of tolterodine, with excellent consistency, over a prolonged 24 hour period. In addition, there is not a need for the use of multiple coatings on a dosage form in order to achieve the observed biopharmaceutical behavior. See, e.g., U.S. Pat. Nos. 6,630,162; 6,770,295; and 6,911,217.

Methods are provided for formulating and manufacturing modified release tolterodine capsule dosage forms for oral delivery. Also provided herein are dosage forms thus produced. Methods are also provided for administering such modified dosage forms to a mammal such as humans. The amount of tolterodine per dosage form can be, as stated conventionally, from about 2 to about 8 mg, including specific intermediate amounts such as 4 mg and 6 mg.

In one aspect, the method comprises the following steps:

a) preparing a mixture of tolterodine and one or more pharmaceutically acceptable excipients to form a tolterodine-excipient mixture and granulating the tolterodine-excipient mixture to produce tolterodine granulates having a granule size of less than 800 μm (e.g., less than 700 μm; less than 600 μm; less than 500 μm; less than 400 μm; less than 300 μm; less than 200 μm; and less than 100 μm);

b) compressing said tolterodine granulates to produce a tolterodine bead or pellet;

c) coating said tolterodine bead or pellet with a release-modifying coating material comprising from about 0.1% to about 30% (e.g., 0.5% to about 10%) by weight of the uncoated bead or pellet, to produce a coated tolterodine bead or pellet;

d) filling an empty capsule with one or more coated tolterodine beads or pellets.

In one aspect, the one or more pharmaceutically acceptable excipients may be selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, glyceryl behenate, and polyols such as mannitol, lactitol, maltitol, sucrose, and sorbitol, or a mixture thereof. The release-modifying coating material may be a material that is known to provide release-modifying characteristics. For example, release-modifying coating materials that provide modified release through diffusion or pH-dependency are useful. Examples include cellulose-based polymers, acrylate polymers (e.g., methacrylate polymers), or waxes or mixtures thereof that may be used in the present invention. Cellulose-based polymers include: ethylcellulose, propylcellulose, hydroxypropylmethylcellulose (e.g., hydroxypropylmethylcellulose generally having a molecular weight of from about 7,000 to about 50,000 including Methocel F50 Premium having a molecular weight of 45,000; methocel E50 Premium LV having a molecular weight of 45,000; Methocel E15 Premium LV having a molecular weight of 30,000; Methocel E6 Premium LV having a molecular weight of 20,000; Methocel E5 Premium LV having a molecular weight of 10,000; and Methocel E3 Premium LV having a molecular weight of 9,000), hydroxymethylpropylcellulose, hydroxypropylcellulose, etc. Acrylate polymers include: methylmethacrylates, methacrylates, etc. The coating composition may also include a plasticizer such as triethylene acetate, triacetene, glycerol, and the like. Alternatively, pH-independent polymers may also be used, such as: methacrylate copolymers with trimethylammonioethylmethacrylate functional groups (sold commercially as Eudragit® RS and Eudragit® RL); neutral polymers of methacrylates (sold commercially as Eudragit® NE40D and Eudragit® NE30D); or mixtures thereof. As used herein, the term “polymer” includes copolymers. In some embodiments, the release-modifying coating materials contemplated by the present invention include ethylcellulose and hydroxypropylmethylcellulose or a mixture thereof.

In another aspect, the method comprises administering the dosage form prepared as above.

In one aspect the invention provides a dosage form of tolterodine prepared according to the methods described herein.

In another aspect, the invention provides an article of manufacture comprising tolterodine prepared in accordance with the methods described herein and accompanying labeling and packaging to enable the article of manufacture to be shipped interstate.

In another aspect, a modified release tolterodine dosage form is provided comprising:

a) a therapeutically effective amount of tolterodine, ranging from about 2 mg to about 8 mg per dosage unit, formulated into one or more pellets comprising said tolterodine and one or more pharmaceutically acceptable excipients;

b) said pellets comprising a pharmaceutically acceptable binder intimately mixed therein with said tolterodine; and

c) a release-modifying film coating substantially completely covering said pellets, wherein said film coating comprises from about 0.1% to about 30% (e.g., 0.5% to about 10%) by weight of the uncoated pellet; wherein said pellets are provided in a capsule dosage form. The capsule dosage form preferably contains a therapeutically effective amount of tolterodine, typically ranging from 2 mg to 8 mg.

In one specific aspect, the release-modifying film coating comprises ethylcellulose. In a preferred mode of this aspect, the release-modifying film coating consists essentially of ethylcellulose, alternatively, the release-modifying film coating consists entirely of ethylcellulose.

In one aspect, the dosage form provides a dissolution profile as following:

about 30% to about 50% of the drug is released by 2 hrs; about 65% to about 90% of the drug is released by 4 hrs; and about 80% to about 100% of the drug is released by 8 hrs, when dissolution test is performed using 900 ml of aqueous medium comprising phosphate buffer at pH 6.8 in a dissolution apparatus using a basket operated at about 37° C., being stirred at a speed of 50 rpm in a USP Type II dissolution testing apparatus.

In another aspect, the invention relates to a modified release tolterodine pharmaceutical composition comprising a plurality of pellets wherein each pellet comprises:
a) a therapeutically effective amount of tolterodine and one or more pharmaceutically acceptable excipients and a binder; and
b) a coating comprising a release-modifying coating material comprising from about 0.1% to about 30% (e.g., 0.5% to about 10%) by weight of the uncoated pellets;
   wherein said pellets have a length of from about 1 mm to about 7 mm and a width of from about 1 mm to about 3 mm; and
   said composition having an in vitro dissolution profile measured in a USP Type II dissolution testing apparatus using 900 ml of aqueous medium comprising phosphate buffer at pH 6.8 in the dissolution apparatus having a basket operated at about 37° C., being stirred at a speed of 50 rpm, where about 30% to about 50% of the tolteridine is released after 2 hours; about 65% to about 90% of the tolteridine is released after 4 hours; and about 80% to about 100% of the tolteridine is released in 8 hours.

The foregoing and other objects and aspects of the present invention are explained in detail in the detailed description and examples set forth below.

### DETAILED DESCRIPTION OF THE INVENTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “tolterodine” refers to a compound also commonly known as tolterodine tartrate. The term also refers to analogs and homologs of tolterodine, including salts in addition to the tartarate salt (e.g., citrate, hydrochloride, etc.), prodrugs, enantiomers and metabolites of tolterodine, as well as mixtures thereof, as dictated by the context of its use.

As used herein, “blood level” may be used interchangeably with terms such as blood plasma concentration, plasma level, plasma concentration, serum level, serum concentration, serum blood level and serum blood concentration.

As used herein, “oral dosage form” and the like refers to a formulation that is ready for administration to a subject through the oral route of administration. Examples of known oral dosage forms, include without limitation, tablets, capsules, caplets, powders, pellets, granules, etc. Such formulations also include multilayered tablets wherein a given layer may represent a different drug. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, “pellet” refers to minitablets having a length of from about 1 mm to about 7 mm and a width of from about 1 mm to about 3 mm. In some aspects, the pellets have a width×length of from about 1 mm×3 mm; about 1 mm×4 mm; about 1 mm×5 mm; about 1 mm×6 mm; about 1 mm×7 mm; about 2 mm×2 mm; about 2 mm×3 mm; about 2 mm×4 mm; about 2 mm×5 mm; about 2 mm×6 mm; about 2 mm×7 mm; or about 3 mm×3 mm. In a preferred aspect, the pellet has a width×length of about 1 mm×3 mm.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable excipient,” “pharmaceutically acceptable carrier,” excipient,” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “modified release” or “release-modifying” as used herein refers to the drug release characteristic that is different from an immediate release. Typically, in an immediate release dosage form, about more than 80% of the drug is released from the dosage form in vitro within about 2 hrs. This release may be measured in terms of dissolution of the drug in the dissolution medium. In one aspect, the release is measured under USP conditions, i.e., where the pH is maintained at 1.2 for 2 hours, followed by a pH of 6.8 for the rest of the time. In another aspect, the release is measured at a pH of 1.2 for the entire period of measurement. Examples of such modified release include sustained release, slow-release, delayed-release, pulsatile release etc., which terms are generally known in the art and to the extent they mean a release other than an immediate release.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

The present invention provides modified release tolterodine dosage forms, e.g., tolterodine oral dosage forms including beads and pellets, with certain desirable in-vitro dissolution properties and in-vivo blood plasma concentrations and pharmacokinetic parameters. In some aspects, the tolterodine dosage forms should be bioequivalent to DETROL® LA, which has a single dose mean area under the curve (AUC_{∞}) of about 13-16 μg·h/L and a mean maximum plasma concentration (Cₘₐₓ) of 1.3 μg/L. See DETROL® LA package insert available at http://www.pfizer.com/files/products/uspi_detrol_la.pdf. As used herein, the term “bioequivalent” means that the values for AUC_{∞} and Cₘₐₓ of the tolterodine dosage forms made according to the invention are between 80% and 125% of the AUC_{∞} and Cₘₐₓ values of commercially available tolterodine tablets (i.e., DETROL® LA tablets).

In one aspect, the invention provides methods for formulating a modified release tolterodine oral dosage form that may be filled into a capsule. The capsule may contain tolterodine and one or more excipients formulated as beads or pellets. A variety of excipients commonly known in the pharmaceutical industry may be used to make the oral dosage form. The tolterodine and excipients together may be formulated into granules. The granules are then either filled into a capsule or are compressed (e.g., using a compression force that ranges from about 10 KN to about 25 KN; or from about 15 KN to about 25 KN; or from about 20 KN to about 25 KN) into pellets which are then coated with a release-modifying composition. Alternatively, the granules are coated with a release-modifying composition and then compressed into a modified-release tolterodine bead or pellet.

In one aspect, the granules may be prepared by the following process. Tolterodine, and one or more inert pharmaceutically acceptable excipients may be mixed intimately to achieve a substantially homogenous mixture. The excipients which may be employed to make the granules are well known to those skilled in the art and include any conventional pharmaceutically acceptable tabletting excipients. Examples of suitable excipients include, but are not limited to, cellulosic materials (e.g., microcrystalline cellulose, hydroxypropylmethyl cellulose (HPMC), hydroxypropyl cellulose (HPC), and the like), xanthan gum, dibasic calcium phosphate dihydrate, starch, sodium starch glycolate, polyvinypyrrolidones (e.g., crospovidone), croscarmellose sodium, magnesium stearate, sugars (e.g., lactose, maltose, dextrose, and the like), maleic acid, wax, colloidal silicon dioxide, talc, and glyceryl behenate, as well as mixtures and various combinations thereof. In some aspects, a mixture comprising HPMC (e.g., HPMC K4M and HPMC K15M), xantham gum, and sugar powder is used to prepare the granules.

The mixing of the excipients and tolterodine can be accomplished by using high shear granulators (mixers, blenders, etc) or rapid mixer granulators. The homogenous mixture may be then processed into granules preferably by wet granulation processes (i.e., using water as the granulating medium) known in the art. Granulation may be performed by the art-known equipment such as high shear granulators, fluid bed granulators, etc. These granules are then optionally dried. The drying process may provide certain advantages such as improvements in content uniformity, ease of handling, etc.

Alternatively, the tolterodine and excipient mixture may be granulated with a non-aqueous solvent. Some examples of non-aqueous solvents include: ethanol, isopropanol, acetone, or methanol or a mixture thereof. In some aspects, the granulating solvent (whether aqueous or nonaqueous) may contain an excipient that serves as a binder. Examples of binders include: ethylcellulose, polyvinylpyrrolidone, hydroxypropylmethyl cellulose (e.g., HPMC E5 LV), starch, gelatin, etc. The amount of this “binder” may range from about 1% to about 25%, e.g., from about 1% to about 15%. In some other aspects, the binder amount may have the following ranges: from about 1% to about 12%; from about 1% to about 10%; from about 1% to about 8%; from about 1% to about 7%; from about 1% to about 6%; from about 1% to about 5%; from about 2% to about 15%; from about 2% to about 12%; from about 2% to about 10%; from about 2% to about 8%; from about 2% to about 6%; from about 2% to about 5%; from about 2% to about 4%; from about 3% to about 15%; from about 3% to about 12%; from about 3% to about 10%; from about 3% to about 8%; from about 3% to about 6%; from about 3% to about 5%; from about 4% to about 12%; from about 4% to about 10%; from about 4% to about 8%; from about 4% to about 6%; from about 5% to about 15%; from about 5% to about 12%; from about 5% to about 10%; from about 5% to about 8%; from about 5% to about 7%. All the above amounts are expressed as w/w % of the oral dosage form.

In some aspects, the particle size of the granules may have the following distribution: about less than 40% of the granules are retained by a #40 mesh; about less than 70% of the particles are retained by a #60 mesh; about less than 90% of the particles are retained by a #80 mesh; and about 100% of the particles are retained by a #200 size mesh. Alternatively, in some aspects, the particle size of the granules may have the following distribution: about less than 60% of the granules are retained by a #40 mesh; about less than 80% of the particles are retained by a #60 mesh; about less than 90% of the particles are retained by a #80 mesh; and about 100% of the particles are retained by a #200 size mesh. Alternatively, in some aspects, the particle size of the granules may have the following distribution: about less than 60% of the granules are retained by a #40 mesh; about less than 90% of the particles are retained by a #60 mesh; and about 100% of the particles are retained by a #200 size mesh.

In some aspects, the granulation process involves the following steps. tolterodine is sifted through a #20 sieve. Excipients are sifted through a #40 sieve. The sifted materials are loaded into a rapid mixer granulator and mixed rapidly for about 5-20 minutes. To this dry mix is added a solution or dispersion of a binder in an aqueous or nonaqueous solvent. Typically, the binder may be any binder that is known the art. Specific examples of binder agents include but are not limited to vinyl polymers, such as polyvinylpyrrolidone, polyvinyl alcohol, and the like; cellulosic polymers, such as HPMC, hydroxyethyl cellulose (HEC), HPC, and the like; acrylic polymers and copolymers such as methacrylic acid copolymers, ethyl acrylate-methylmethacrylate copolymers, and the like; natural or synthetic gums, such as guar gum, arabic gum, xanthan gum, and the like; proteins or carbohydrates, such as gelatin, pectin, and the like; and mixtures thereof. In some aspects, ethylcellulose, polyvinylpyrrolidone, or mixtures thereof are preferred binding agents. One specific binder is polyvinylpyrrolidone K-30 and the nonaqueous solvent is isopropyl alcohol.

Suitable binder dispersions may include conventional pharmaceutically acceptable binder agents solubilized in a suitable solvent such as ethanol, isopropyl alcohol, or, in some cases, acetone.

Suitable solvents for solubilizing the binder agents include solvents which are capable of substantially completely solubilizing the specific binder agent(s) selected and which are pharmaceutically and biologically acceptable for ingestion. Suitable solvents will be readily determinable by those skilled in the art. Water is currently the preferred solvent for solubilizing the binder agent. However, other examples of suitable solvents will be appreciated by those skilled in the art and are contemplated by the methods of the present invention.

The binder solution should be of sufficient viscosity (e.g., below 300 cP) to enable the wetting of the dry tolterodine excipient mixture by any suitable wetting technique known to those skilled in the art. For example, the powder may be wetted with the binder solution by rotating the powder in a bath containing the binder solution. Alternatively, the powder may be suitably wetted by manual application of the binder dispersion by layering the binder solution over the powder as the powder is rotating in a conventional coating pan. Alternatively, the powder may be wetted by spraying the binder dispersion on the powder. In one aspect, the wetting step is carried out using conventional automated pan coating equipment wherein the powder is sprayed with the binder dispersion while rotating in the pan. However, the granulation step is accomplished in one single pot (i.e., the rapid mixer granulator), without the need to transfer the dry mix into a separate granulator, if it is conducted in the rapid mixer granulator.

In one aspect, tolterodine granulates are optionally dried prior to, e.g., compression into pellets, to substantially remove any residual solvents. The granulates are then compressed into pellets. Alternatively, the granulates are coated with a release-modifying coating which are then compressed into modified release pellets. In some aspects, each of the bead or pellet may weigh about 20 mg and contain about 1 mg of tolterodine. Thus, in one aspect, a capsule providing 4 mg dose of tolterodine may be obtained by filling an empty size “2” hard gelatin capsule with four of the pellets. In some aspects, the capsules are prepared for other doses with proportionate amounts of pellets.

The pellets thus obtained (comprising either coated or uncoated granulates) may optionally be coated with a release-modifying coating that substantially surrounds the pellets. The coating material for coating either the granules or the compressed bead/pellet can comprise the same or different material and may be present in the same or different concentrations. Coating may be accomplished by either aqueous or solvent-based techniques. The coating equipment is known in the art. For example, conventional coating pans or automatic fluid bed coaters may be used.

The release-modifying coating material comprises generally a coating material that is known to provide release-modifying characteristics. For example, release-modifying polymers that provide modified release through diffusion or pH-dependency are useful. Examples include cellulose-based polymers, acrylate polymers (e.g., methacrylate polymers), or waxes or mixtures thereof that may be used in the present invention. Cellulose-based polymers include: ethylcellulose, propylcellulose, hydroxypropylmethylcellulose (e.g., hydroxypropylmethylcellulose generally having a molecular weight of from about 7,000 to about 50,000 including Methocel F50 Premium having a molecular weight of 45,000; methocel E50 Premium LV having a molecular weight of 45,000; Methocel E15 Premium LV having a molecular weight of 30,000; Methocel E6 Premium LV having a molecular weight of 20,000; Methocel E5 Premium LV having a molecular weight of 10,000; and Methocel E3 Premium LV having a molecular weight of 9,000), hydroxymethylpropylcellulose, hydroxypropylcellulose, etc. Acrylate polymers include: methylmethacrylates, methacrylates, etc. The coating composition may also include a plasticizer such as triethylene acetate, triacetene, glycerol, and the like. Alternatively, pH-independent polymers may also be used, such as: methacrylate copolymers with trimethylammonioethylmethacrylate functional groups (sold commercially as Eudragit® RS and Eudragit® RL); neutral polymers of methacrylates (sold commercially as Eudragit® NE40D and Eudragit® NE30D); or mixtures thereof. As used herein, the term “polymer” includes copolymers.

The amount of release-modifying coating material may range from about 0.1% to about 30% by weight of the uncoated tablet or granule. In some aspects, the coating may range from: about 0.5% to about 10%; from about 0.5% to about 2%; from about 0.5% to about 8%; from about 0.5% to about 5%. In one specific aspect, the coating material comprises a mixture of Eudragit RS 100, hydroxypropylcellulose, and HPMCE5LV in a ratio of about 90:5:5, or 90:3:7, or 80:5:15 or 85:5:10. In one aspect, the Eudragit RS100/HPC/HPMC coating composition comprised of about 7% by weight of the uncoated pellet. In some aspects, the above coating material also comprises a plasticizer, such as triethylcitrate or talc or a mixture thereof in a ratio of 1:5 to 5:1 totaling from about 1% to about 5% by weight of the uncoated pellet.

In addition to the foregoing, the compositions of the present invention may also include other excipients such as lubricants (e.g., stearate salts of calcium, magnesium, and zinc, as well as titanium dioxide, silicon dioxide, talc, kaolin, and glycerol monostearate), flow promoting agents (e.g., silicon dioxide), plasticizers (e.g., triethylcitrate, and other citrate esters, diethyl phthalate, tributyl citrate, and dibutyl sebacate), anti-sticking agents, natural and synthetic flavorings and natural and synthetic colorants. Specific examples of additional excipients include polyethylene glycol, a wax, polyvinylpyrrolidone, talc, magnesium stearate, glyceryl behenate, stearic acid, titanium dioxide, and polyols such as mannitol, lactitol, maltitol, sucrose, and sorbitol. In some preferred embodiments, preferred additional excipients are magnesium stearate and/or stearic acid.

Additional Agents

The present invention also provides modified release formulations of tolterodine in combination with other pharmaceutically active agents. Examples of such additional agents include: other antimuscarinic agents, psychotherapeutic agents. Examples of antimuscarinic agents include: oxybutynin, hyoscamine, bethanechol, etc. Examples of psychotherapeutic agents include: antidepressants, anti-anxiety agents, antipsychotics, etc. Examples of antidepressants include: buspoirone, bupropion, thioridazine, venlafaxine, fluoxetine, paroxetine, etc. Examples of anti-anxiety agents include: diazepam, lorazepam, trazodone, alprazolam, etc. Examples of antipsychotics include: loxitane, thothixene, prochlorperazine, risperidone, olanzapine, etc. It should be noted that one or more of these additional ingredients may be present in an immediate release form, in a modified release form, or in a pulsatile release form or in a fast disintegrating form. The additional agents may be present in an intimate mixture with the tolterodine or separated by an additional barrier layer.

The formulations of the present invention are provided in the form of capsules wherein the beads or pellets of the present invention is used to fill in a conventional hard or soft gelatin capsule. Encapsulation within a soft-gelatin capsule is also achievable with conventional techniques. Alternatively, the beads or pellets of the invention can be compressed into larger tablets (e.g., tablets having a diameter of 9-10 mm) by using conventional techniques that are well-known in the art, which may be suitable for administration to the patient, or these tablets are then used to fill in the capsules for administration.

In one specific aspect, the compositions of the present are combinable in smaller doses to achieve the therapeutic benefit of a higher dose. For example, two 2 mg capsules may be administered together to provide the therapeutic benefit of a 4 mg capsule. Similarly, a 2 mg and a 4 mg composition of the present invention may be combined to provide the benefit of a 6 mg dose.

Additionally, the present invention also provides methods of achieving desired therapeutic benefit from tolterodine therapy by administering to the subject the oral dosage forms described herein prepared according to the presently disclosed methods.

### EXAMPLES

The following examples are provided to illustrate the present invention, and should not be construed as limiting thereof. All percentages are in percent by weight of the formulation unless otherwise indicated. Disintegration tests are carried out according to the standard procedures set forth in the United States Pharmacopoeia for testing the disintegration of capsules.

### Example 1

2.6 g of Tolterodine tartarate, 20 g of microcrystalline cellulose powder, 25.4 g of lactose, were mixed and passed through # 22 mesh. Separately, 1.5 g of plasdone K29/32 was dissolved in 30 ml of Isopropyl alcohol. The tolterodine mixture was granulated with the isopropyl alcohol solution and the blend was passed through #18 and kept for air drying.

0.5 g of magnesium stearate were passed through #30 and added to the above granulation mixture. The lubricated blend was compressed into 3.0 mm size pellets.

The pellets were then coated with a coating composition comprising: 7.98 g of Eudragit RS100 (Degussa), 0.42 g of HPC (Klucell EF), 1.68 g of triethylcitrate in 1.68 g of methanol. The coated pellets were filled into No. ‘2’ size hard gelatin capsules.

Results: Dissolutions were conducted using USP— Type II apparatus, operated at 100 RPM, using 900 ml of the medium. The medium had a pH value of 6.8 comprising phosphate buffer.

| Time points | pH medium | % RELEASE |
|---|---|---|
| 1 hr | 6.8 | 17.28 |
| 2 hr | 6.8 | 42.63 |
| 3 hr | 6.8 | 58.86 |
| 4 hr | 6.8 | 68.03 |
| 6 hr | 6.8 | 77.03 |
| 7 hr | 6.8 | 77.42 |

### Example 2

2.6 g of Tolterodine tartarate, 20 g of microcrystalline cellulose powder, 25.4 g of lactose, were mixed and passed through # 22 mesh. Separately, 1.5 g of plasdone K29/32 was dissolved in 30 ml of Isopropyl alcohol. The tolterodine mixture was granulated with the isopropyl alcohol solution and the blend was passed through #18 and kept for air drying.

0.5 g of magnesium stearate was passed through #30 and added to the above granulation mixture. The lubricated blend was compressed into 3.0 mm size pellets.

The pellets (comprising 120 grams of tolterodine) were then coated with a coating composition comprising: 7.98 g of Eudragit RS100 (Degussa), 0.42 g of HPC (Klucell EF), 1.68 g of triethylcitrate in 420 ml of methanol. The coated pellets were filled into No. ‘2’ size hard gelatin capsules.

### Results:

Dissolution studies: Same conditions as in Example 1

| Time points | pH media | % RELEASE |
|---|---|---|
| 1 hr | 6.8 | 16.86 |
| 2 hr | 6.8 | 34.31 |
| 3 hr | 6.8 | 47.01 |
| 4 hr | 6.8 | 55.26 |
| 6 hr | 6.8 | 66.96 |
| 7 hr | 6.8 | 71.51 |

### Example 3

Part-A: 0.65 g of Tolterodine tartarate, 5.48 g of sugar powder (# 120 screened), were mixed and the mixture was passed through # 22 mesh.

Part-B: 0.63 g of plasdone K29/32 was dissolved in 10 ml of Isopropyl alcohol.

### Granulation and Drying:

Granulation was performed as per Example 1.

### Lubrication:

0.13 g of magnesium stearate was passed through #30 and was added to the above granulation mixture. The lubricated blend was compressed into 3.0 mm size pellets, which were then filled into a No. ‘2’ size hard gelatin capsule.

### Results:

### Dissolution Studies: Same as in Example 1

| Time points | pH Media | % RELEASE |
|---|---|---|
| 1 hr | 6.8 | 13.97 |
| 2 hr | 6.8 | 28.89 |
| 3 hr | 6.8 | 32.72 |
| 4 hr | 6.8 | 47.76 |
| 6 hr | 6.8 | 68.84 |
| 7 hr | 6.8 | 71.81 |

### Example 4

Part-A: 0.65 g of Tolterodine tartarate, 5.48 g of sugar powder (# 120 screened) were mixed and the mixture was passed through # 22 mesh. ***

Part-B: 0.63 g of plasdone K29/32 was dissolved in 10 ml of Isopropyl alcohol.

### Granulation and Drying:

Granulation was performed as per Example 1.

### Lubrication:

0.13 g of magnesium stearate was passed through #30 and was added to the above granulation mixture. The lubricated blend was compressed into 3.0 mm size pellets and were filled into No. ‘2’ size hard gelatin capsule.

### Results

### Dissolution Studies: Same as in Example 1

| TIME | pH | % RELEASE |
|---|---|---|
| 1 hr | 6.8 | 21.09 |
| 2 hr | 6.8 | 49.41 |
| 3 hr | 6.8 | 71.19 |
| 4 hr | 6.8 | 74.08 |
| 6 hr | 6.8 | 86.72 |
| 7 hr | 6.8 | 89.22 |

### Example 5

Part-A: 0.65 g of Tolterodine tartarate, 0.31 g of methyl cellulose MP-814 pharm, 2.04 g of lactose, 5.0 g of sugar powder (# 120 screened) were mixed and the mixture was passed through # 22 mesh.

Part-B: 0.63 g of plasdone K29/32 was dissolved in 10 ml of Isopropyl alcohol.

### Granulation and Drying:

Granulation and drying was performed as per Example 1.

### Lubrication:

0.13 g of magnesium stearate was passed through #30 and was added to the above granulation mixture. The lubricated blend was compressed into 3.0 mm size pellets and were filled into a No. ‘2’ size hard gelatin capsule.

### Results:

Dissolution Studies: Conditions were the Same as in Example 1.

| Time points | pH media | % RELEASE |
|---|---|---|
| 1 hr | 6.8 | 12.08 |
| 2 hr | 6.8 | 41.22 |
| 3 hr | 6.8 | 61.78 |
| 4 hr | 6.8 | 71.76 |
| 6 hr | 6.8 | 80.27 |
| 7 hr | 6.8 | 82.07 |

### Example 6

1.04 g of Tolterodine tartarate, 8 g of microcrystalline cellulose powder, 10.08 g of lactose were mixed and the mixture was passed through # 22 mesh. Separately, 0.6 gm of plasdone K29/32 was dissolved in 20 ml of Isopropyl alcohol. The tolterodine mixture was granulated with the isopropyl alcohol solution and the blend was passed through #18 and kept for air drying.

0.2 g of magnesium stearate was passed through #30 and was added to the above granulation mixture. The lubricated blend was then compressed into 3.0 mm size pellets. These pellets were then coated with a composition as follows: 1.53 gms of Eudragit RS100, 0.05 gms of HPC and 0.12 gms of HPMC E5LV per tablet (90:3:7) comprising a 7% coating. The composition also contained 0.34 mg of triethylcitrate, and 0.48 mg of talc as plasticizers.

Results: Dissolutions were conducted as described in Example 1.

| Time points | pH media | % RELEASE |
|---|---|---|
| 1 hr | 6.8 | 16.19 |
| 2 hr | 6.8 | 49.83 |
| 3 hr | 6.8 | 76.24 |
| 4 hr | 6.8 | 90.94 |
| 6 hr | 6.8 | 100.98 |
| 7 hr | 6.8 | 114.2 |

### Example 7

A controlled release tolterodine tartrate pellet was developed having a core that uses high molecular weight HPMC. As hydration takes place, it forms a highly viscous gel (hydrogel) that initiates and regulate release of tolterodine tartrate.

A tolterodine-excipient mixture was made by mixing 6.5 g of tolterodine tartrate, 25 g of hydroxy propyl methyl cellulose K4M, 12.5 g of hydroxy propyl methyl cellulose K15M, 18.8 g of xanthan gum, and 54.8 g of sugar powder. The resulting mixture was passed through a # 25 mesh. In a separate container, 6.3 g of povidone was dissolved in 25 ml of isopropyl alcohol under continuous stirring until clear solution was formed. This formed the binder solution. The tolterodine-excipient mixture was granulated using a rapid mixing granulator using the binder solution. The wet mass was passed through a #8 mesh and kept for drying. After complete drying, the dry-granulate mixture was passed through a #25 screen. Magnesium stearate (1.3 g) was passed through #30 mesh and added to the dry-granulate mixture and blended for 10 minutes in a mixer. The lubricated blend was compressed using 3.0 mm punches to give 20 mg pellets with hardness of 2.62 Kpa, thickness of 2.26 mm, and friability of NMT 1.0%.

Dissolution testing of the pellets was conducted using USP-Type I apparatus, at 100 RPM, using 900 ml of 6.8 pH phosphate buffer at 37° C.

| Time points | Dissolution Release |
|---|---|
| (hrs) | Profile (%) |
| 1 | 41.76 |
| 2 | 63.18 |
| 3 | 75.47 |
| 4 | 87.0 |
| 6 | 87.13 |
| 7 | 89.93 |

### Example 8

A controlled release tolterodine tartrate pellet was developed having a core that uses high molecular weight HPMC. As hydration takes place, it forms a highly viscous gel (hydrogel) that initiates and regulate release of tolterodine tartrate.

A tolterodine-excipient mixture was made by mixing 6.5 g of tolterodine tartrate, 12.5 g of hydroxy propyl methyl cellulose K4M, 25.0 g of hydroxy propyl methyl cellulose K15M, 18.8 g of xanthan gum, and 54.8 g of sugar powder. The resulting mixture was passed through a # 25 mesh. In a separate container, 6.3 g of povidone was dissolved in 25 ml of isopropyl alcohol under continuous stirring until clear solution was formed. This formed the binder solution. The tolterodine-excipient mixture was granulated using a rapid mixing granulator using the binder solution. The wet mass was passed through a #8 mesh and kept for drying. After complete drying, the dry-granulate mixture was passed through a #25 screen. Magnesium stearate (1.3 g) was passed through #30 mesh and added to the dry-granulate mixture and blended for 10 minutes in a mixer. The lubricated blend was compressed using 3.0 mm punches to give 20 mg pellets with hardness of 2.62 Kpa, thickness of 2.26 mm, and friability of NMT 1.0%.

Dissolution testing of the pellets was conducted using USP-Type I apparatus, at 100 RPM, using 900 ml of 6.8 pH phosphate buffer at 37° C.

| Time points | Dissolution Release |
|---|---|
| (hrs) | Profile (%) |
| 1 | 47.05 |
| 2 | 71.15 |
| 3 | 87.60 |
| 4 | 98.37 |
| 6 | 100.5 |
| 7 | 101.05 |

### Example 9

A controlled release tolterodine tartrate pellet was developed having a core that uses high molecular weight HPMC. As hydration takes place, it forms a highly viscous gel (hydrogel) that initiates and regulate release of tolterodine tartrate.

A tolterodine-excipient mixture was made by mixing 6.5 g of tolterodine tartrate, 3.1 g methyl cellulose, 25.0 g of hydroxy propyl methyl cellulose K4M, 12.5 g of xanthan gum, 50.0 g of sugar powder, and 20.4 g lactose. The resulting mixture was passed through a # 25 mesh. In a separate container, 6.3 g of povidone was dissolved in 25 ml of isopropyl alcohol under continuous stirring until clear solution was formed. This formed the binder solution. The tolterodine-excipient mixture was granulated using a rapid mixing granulator using the binder solution. The wet mass was passed through a #18 mesh and kept for drying. After complete drying, the dry-granulate mixture was passed through a #25 screen. Magnesium stearate (1.3 g) was passed through #30 mesh and added to the dry-granulate mixture and blended for 10 minutes in a mixer. The lubricated blend was compressed using 3.0 mm punches to give 20 mg pellets with hardness of 2.62 Kpa, thickness of 2.26 mm, and friability of NMT 1.0%.

Dissolution testing of the pellets was conducted using USP-Type I apparatus, at 100 RPM, using 900 ml of 6.8 pH phosphate buffer at 37° C.

| Time points | Dissolution Release |
|---|---|
| (hrs) | Profile (%) |
| 1 | 56.84 |
| 2 | 77.8 |
| 3 | 92.7 |
| 4 | 98.8 |
| 6 | 100.8 |
| 7 | 101.2 |

### Example 10

A controlled release tolterodine tartrate pellet was developed having a core that uses highly viscous water soluble and swellable gums. As hydration takes place, it forms high viscous gels (hydrogels) that initiates and regulate release of tolterodine tartrate in the core.

A tolterodine-excipient mixture was made by mixing 6.5 g of tolterodine tartrate, 62.5 g of xanthan gum, 12.5 g guar gum, and 36.0 g of sugar powder. The resulting mixture was passed through a # 25 mesh. In a separate container, 6.3 g of povidone was dissolved in 25 ml of isopropyl alcohol under continuous stirring until clear solution was formed. This formed the binder solution. The tolterodine-excipient mixture was granulated using a rapid mixing granulator using the binder solution. The wet mass was passed through a #8 mesh and kept for drying. After complete drying, the dry-granulate mixture was passed through a #25 screen. Magnesium stearate (1.3 g) was passed through #30 mesh and added to the dry-granulate mixture and blended for 10 minutes in a mixer. The lubricated blend was compressed using 3.0 mm punches to give 20 mg pellets with hardness of 2.62 Kpa, thickness of 2.26 mm, and friability of NMT 1.0%.

Dissolution testing of the pellets was conducted using USP-Type I apparatus, at 100 RPM, using 900 ml of 6.8 pH phosphate buffer at 37° C.

| Time points | Dissolution Release |
|---|---|
| (hrs) | Profile (%) |
| 1 | 48.66 |
| 2 | 74.10 |
| 3 | 82.01 |
| 4 | 95.67 |
| 6 | 98.3 |
| 7 | 99.6 |

### Example 11

A controlled release tolterodine tartrate pellet was developed having a core that uses microcrystalline cellulose powder, lactose granulate with less molecular weight hydroxy propyl methyl cellulose. The pellets were coated with diffusion layer comprising a combination of water insoluble polymer, water soluble and channel forming polymers. The coating regulates release of tolterodine tartrate.

A tolterodine-excipient mixture was made by mixing 13.0 g of tolterodine tartrate, 101.0 g of microcrystalline cellulose powder, and 126.0 g lactose. The resulting mixture was passed through a # 25 mesh. In a separate container, 7.5 g of hydroxypropylmethyl cellulose E5 LV was dissolved in 125 ml of methanol under continuous stirring until clear solution was formed. This formed the binder solution. The tolterodine-excipient mixture was granulated using a rapid mixing granulator using the binder solution. The wet mass was passed through a #18 mesh and kept for drying. Magnesium stearate (2.5 g) was passed through #30 mesh and added to the dry-granulate mixture and blended for 10 minutes in a mixer. The lubricated blend was compressed using 3.0 mm punches to give 20 mg pellets with hardness of 2.62 Kpa, thickness of 2.26 mm, and friability of NMT 1.0%.

A controlled release coating solution was prepared using 14.40 g of poly(ethylacrylate, methylmethacrylate, trimethylammonium ethyl methacrylate chloride) commercially available as Eudragit® RS 100 (Degussa), 0.48 g of hydroxypropyl cellulose, 1.12 g of hydroxypropylmethyl cellulose E5 LV, 1.60 g of talc, 1.60 g of triethylcitrate in 750 ml of methanol and 50 ml of water. The pellets were coated with the coating solution using a fluid bed processor with wurster mode (bottom spray).

Dissolution testing of the pellets was conducted using USP-Type I apparatus, at 100 RPM, using 900 ml of 6.8 pH phosphate buffer at 37° C.

| Time interval | |
|---|---|
| (hrs) | % Release |
| 1 | 19.64 |
| 2 | 46.19 |
| 3 | 66.59 |
| 4 | 73.3 |
| 6 | 72.19 |
| 7 | 79.81 |

The release percentage of the tolterodine tartrate in this Example and in Examples 7-9, was measured using the conditions required by the United States Food and Drug Administration for this drug. The dissolution was performed using USP apparatus I with 900 ml of 6.8 pH phosphate buffer at 100 RPM at 37° C. The dosage forms tested provide a dissolution profile that can be generally summarized as: about 30% to about 50% of the drug is released by 2 hrs; about 65% to about 90% of the drug is released by 4 hrs; and about 80% to about 100% of the drug is released by 8 hrs.

The release percentage of the tolterodine was measured using High Pressure Liquid Chromatography (HPLC) using the following parameters:

Column: C-18, 250×4.6 mm, 5 μm column;

Phosphate mobile phase: Methanol with 6.8 pH phosphate buffer;

Flow rate: 1.0 ml/min;

Wavelength: 215 nm; and

Temperature: ambient.

### Example 12

### Stability Testing

The tolterodine tartrate delayed release capsules of Example 10 were charged under accelerated conditions (i.e., 40° C.±2° C. and Relative humidity 75±5%) to check the stability of the formulation. Release percentage of the tolterodine tartrate controlled release capsules were measured in the same manner described in Example 10.

% Release

| | |
|---|---|
| Time interval | 0 Month |

| (Hour) | Medium | (Initial) | 1^{st} Month |
|---|---|---|---|
| 1^{st} | 6.8 pH | 19.64 | 14.52 |
| 2^{nd} | 6.8 pH | 46.19 | 40.42 |
| 3^{rd} | 6.8 pH | 66.59 | 60.68 |
| 4^{th} | 6.8 pH | 73.3 | 74.32 |
| 6^{th} | 6.8 pH | 72.19 | 86.30 |
| 7^{th} | 6.8 pH | 79.81 | 89.54 |

### Example 13

A tolterodine-excipient mixture was made by mixing 3.9 g of tolterodine tartrate, 30.30 g of microcrystalline cellulose powder, and 37.80 g lactose. The resulting mixture was mixed in a high shear granulator for ten minutes. In a separate container, 2.25 g plasdone k29/32 USP was dissolved in 20 ml isopropyl alcohol under continuous stirring until a clear solution was formed. This formed the binder solution. The tolterodine-excipient mixture was granulated using a rapid mixing granulator using the binder solution. The binder solution was added at a rate of 2 ml/min. The granulated material was dried using a tray dryer at a temperature of 50° C. The granules were dried until the moisture content was below 1% (observed content after drying is 0.78%. The dried granules were passed through a #25 mesh. Magnesium stearate (0.75 g) was passed through #30 mesh and added to the dry-granulate mixture and blended for 10 minutes in a mixer. The lubricated blend was compressed using 3.0 mm punches to give 20 mg pellets, and thickness of 2.8 mm.

A controlled release coating solution was prepared using 14.40 g of Eudragit® RS 100 (Degussa), 0.4 g of hydroxypropyl cellulose (Klucel EF), 1.12 g of hydroxypropylmethyl cellulose E5 LV (90:3:7 Eudragit:Klucel EF:HPMC E5 LV), 4.00 g of talc, 3.21 g of triethylcitrate in 760 ml of methanol and 50 ml of water (95:5 methanol:water). The pellets were coated with the coating solution using a fluid bed processor with wurster mode (bottom spray).

Dissolution testing of the pellets with varying weight percent coatings was conducted using USP-Type I apparatus, at 100 RPM, using 900 ml of 6.8 pH phosphate buffer at 37° C. and compared to the dissolution of Detrol® LA.

| Time | % Release | % Release | % Release | % Release | % Release |
|---|---|---|---|---|---|
| (hours) | Detrol ® LA | 4% coated | 5% coated | 6% coated | 7% coated |
| 1 | 16.39 | 37.77 | 28.31 | 20.89 | 16.19 |
| 2 | 45.48 | 64.95 | 58.53 | 49.71 | 48.83 |
| 3 | 69.62 | 78.95 | 75.5 | 71.79 | 76.24 |
| 4 | 81.78 | 91.7 | 88.95 | 85.61 | 90.94 |
| 6 | 92.89 | 98.16 | 98.68 | 96.74 | 100.93 |
| 7 | 97.31 | 99.92 | 102.5 | 101.9 | 114.2 |

Dissolution testing of the pellets (7% coat) was conducted using USP-Type I apparatus, at 100 RPM, using 900 ml at 1.2 pH (simulated gastric fluid without pepsin) at 37° C. and compared to the dissolution of Detrol® LA.

| Time | % Release | % Release |
|---|---|---|
| (hours) | Detrol ® LA | 7% Coated |
| 1 | 11.94 | 4.31 |
| 2 | 15.57 | 10.38 |
| 3 | 17.48 | 15.39 |
| 4 | 23.89 | 21.77 |
| 6 | 34.96 | 30.02 |
| 7 | 40.38 | 35.11 |

Dissolution testing of the pellets (7% coat) was conducted using USP-Type I apparatus, at 100 RPM, using 900 ml at 4.5 pH (phosphate buffer) at 37° C. and compared to the dissolution of Detrol® LA.

| | | % |
|---|---|---|
| | % Release | Release |
| Time | Detrol ® | 7% |
| (hours) | LA | Coated |
| 1 | 23.21 | 15.95 |
| 2 | 53.5 | 44.33 |
| 3 | 77.3 | 64.01 |
| 4 | 89.49 | 78.15 |
| 6 | 100.09 | 92.6 |
| 7 | 100.2 | 96.99 |

Dissolution testing of the pellets (7% coat) was conducted using USP-Type I apparatus, at 100 RPM, using 900 ml of water at 37° C. and compared to the dissolution of Detrol® LA.

The foregoing is illustrative of the present invention and is not to be construed as limiting thereof. The invention is defined by the following claims, with equivalents of the claims to be included therein.

What is claimed is:

1. A method of preparing a modified release tolterodine pharmaceutical composition comprising:
a) preparing a mixture of tolterodine and one or more pharmaceutically acceptable excipients to form a tolterodine-excipient mixture and granulating the tolterodine-excipient mixture in the presence of a binder to produce tolterodine granulates having a granule size of less than 800 μm;
b) compressing said tolterodine granulates into a tolterodine pellet having a length of from about 1 mm to about 7 mm and a width of from about 1 mm to about 3 mm; and
c) coating said tolterodine pellet with a release-modifying coating material comprising from about 0.1% to about 30% by weight of the uncoated pellet, to produce a coated tolterodine pellet; and
d) filling an empty hard gelatin capsule with one or more coated tolterodine pellets or compressing a plurality of said coated pellets into a tablet.

2. The method of claim 1, wherein said pellet has a width of about 1 mm and a length of about 3 mm.

3. The method of claim 1, wherein the pharmaceutically acceptable excipient is microcrystalline cellulose, dibasic calcium phosphate dihydrate, starch, sodium starch glycolate, crospovidone, croscarmellose sodium, magnesium stearate, maleic acid, colloidal silicon dioxide, talc, glyceryl behenate, mannitol, lactitol, maltitol, sucrose, sorbitol, or mixtures thereof.

4. The method of claim 1, wherein the pharmaceutically acceptable excipient is polyvinylpyrrolidone.

5. The method of claim 1, wherein the binder is ethylcellulose, povidone, or hydroxypropylmethyl cellulose, and comprises from about 1% t about 5% by weight of the mixture.

6. The method of claim 1, wherein the release-modifying coating material is a cellulose-based polymer, acrylate polymer, a wax, a gum or mixtures thereof.

7. The method of claim 6, wherein the acrylate polymer is a pH-independent methacrylate polymer.

8. The method of claim 6, wherein the release-modifying coating material comprises ethylcellulose, hydroxypropylmethylcellulose, methylmethacrylate, methylethacrylate, or a mixture thereof.

9. The method of claim 8, wherein the hydropropylmethylcellulose has a molecular weight of from about 7,000 to about 50,000.

10. The release-modifying coating material of claim 1 further comprising a plasticizing agent.

11. A method of administering tolterodine therapy to a subject in need thereof, comprising:
administering a therapeutically effective amount of the tolterodine composition of claim 1.

12. The method of claim 11, wherein said formulation further comprises an additional active comprising an additional antimuscarinic agent, a psychotherapeutic agent, or a mixture thereof.

13. The method of claim 12, wherein said additional active agent is buspoirone, bupropion, thioridazine, venlafaxine, fluoxetine, paroxetine, diazepam, lorazepam, trazodone, alprazolam, loxitane, thothixene, prochlorperazine, risperidone, olanzapine, or a mixture thereof.

14. A modified release tolterodine pharmaceutical composition comprising a plurality of pellets wherein each pellet comprises:
a) a therapeutically effective amount of tolterodine and one or more pharmaceutically acceptable excipients and a binder; and
b) a coating comprising a release-modifying coating material comprising from about 0.1 to about 30% by weight of the uncoated pellets;
wherein said pellets have a length of from about 1 mm to about 7 mm and a width of from about 1 mm to about 3 mm; and
said composition having an in vitro dissolution profile measured in a USP Type II dissolution testing apparatus using 900 ml of aqueous medium comprising phosphate buffer at pH 6.8 in the dissolution apparatus having a basket operated at about 37° C., being stirred at a speed of 50 rpm, where about 30% to about 50% of the tolteridine is released after 2 hours; about 65% to about 90% of the tolteridine is released after 4 hours; and about 80% to about 100% of the tolteridine is released in 8 hours.

15. The composition of claim 14, wherein said pellet has a width of about 1 mm and a length of about 3 mm.

16. The composition of claim 14, wherein the pharmaceutically acceptable excipient is microcrystalline cellulose, dibasic calcium phosphate dihydrate, starch, sodium starch glycolate, crospovidone, croscarmellose sodium, magnesium stearate, maleic acid, colloidal silicon dioxide, talc, glyceryl behenate, mannitol, lactitol, maltitol, sucrose, sorbitol, or mixtures thereof.

17. The composition of claim 14, wherein the pharmaceutically acceptable excipient is polyvinylpyrrolidone.

18. The composition of claim 14, wherein the binder is ethylcellulose, povidone, or hydroxypropylmethyl cellulose, and comprises from about 1% t about 5% by weight of the mixture.

19. The composition of claim 14, wherein the release-modifying coating material comprises a cellulose-based polymer, acrylate polymer, a wax, a gum or mixtures thereof.

20. The composition of claim 19, wherein the acrylate polymer is a pH-independent methacrylate polymer.

21. The composition of claim 19, wherein the release-modifying coating material is ethylcellulose, hydroxypropylmethylcellulose, methylmethacrylate, methylethacrylate, or a mixture thereof.

22. The composition of claim 21, wherein the hydropropylmethylcellulose has a molecular weight of from about 7,000 to about 50,000.

23. The composition of claim 14, wherein the release-modifying coating material further comprises a plasticizing agent.
